(19) United States
(12) Patent Application Publication
(10) Pub. No.: US 2019/0269608 A1
(43) Pub. Date: Sep. 5, 2019

(54) PET CHEW WITH DENTAL HEALTH FUNCTION

(71) Applicant: INTELLIGENCE ENGINEERING INC., ROAD TOWN (VG)

(72) Inventors: CHRISTOPHER KENNETH CLEVERS, ROAD TOWN (VG); TOMMY CHI-KIN WONG, KOWLOON BAY (HK); TIN-HAN CHIEN, TAINAN CITY (TW)

(21) Appl. No.: 15/909,981

(22) Filed: Mar. 1, 2018

(51) Int. Cl. A61K 8/99; A61K 8/02; A61K 8/60; A61K 8/98; A61K 8/64; A61K 8/73; A61K 8/92; A61K 8/65; A61Q 11/00; A61K 8/97; A01K 15/02

(52) U.S. Cl. CPC A61K 8/99; A61K 2800/92; A61K 8/60; A61K 8/98; A61K 8/64; A61K 8/733; A61K 8/927; A61K 8/732; A61K 8/65; A61Q 11/00; A61K 8/97; A61K 8/731; A01K 15/026; A61K 2800/87; A61K 8/0216

(57) ABSTRACT

A pet chew with a dental health function includes a carrier and plural dental health members disposed on the surface of interior of the carrier. The dental health members include probiotic, prebiotic and adhesive. The probiotic is a human intestinal probiotic group, and a human beneficial probiotic. Since pets like molar, the chew induces a pet to chew for more than 3 minutes, and the probiotic and prebiotic can clean the pet's teeth during the long chewing time. An adhesive is mixed into the probiotic and prebiotic, so that a large amount of probiotic and prebiotic is adhered into the pet's mouth. The prebiotic promotes the colonization of the probiotic, forms advantageous probiotic groups, and inhibits the breeding of germs in the pet's mouth, and the growth of human harmful germs, so as to achieve the effects of maintaining the pet's oral health and eliminating bad breath.

### FIELD OF INVENTION

The present disclosure relates to the field of pet chew toys, in particular to a pet chew with a dental health function.

### BACKGROUND OF INVENTION

### 1. Description of the Related Art

As economy grows, more and more pets become a part of our daily life. During the feeding process, food residues are adhered to the pet's teeth easily, particularly for the pets such as dogs and cats. Tartar and dental calculus are accumulated, and bacteria are bred, and periodontal disease, tooth fracture, mandibular fracture and oral inflammation are resulted. At present, it is believed that periodontitis is mainly caused by bacteria such as porphyromonas gingivalis, tannerella forsythia, and fusobacterium nucleatum, and the microbial balance in a pet's mouth is no longer maintained, so that related oral diseases occur, and various oral diseases further aggravates the imbalance of microbial flora, and helping germs to breed. Taking pet dogs for example, reports show that the incidence of periodontal disease of a 2-8 year-old pet dog is 60%-80% and increases with age. After a pet has suffered from oral diseases, the main treatment methods include cleaning the pet's mouth, removing tartar and calculus by ultrasonic waves, and adopting board-spectrum anti-inflammatory antibiotics. However, it takes much time to clean a pet's mouth. Although using antibiotics for the treatment has a specific efficacy in short time, yet the antibiotics may cause the imbalance of the original microbial flora in the pet's mouth, and ultimately exacerbate the pet's oral problem. In addition, if the pet's mouth carries germs or the pet suffers from an oral disease, not just the pet's health will be affected only, but the pet owner's health particularly for infants, babies or elderly people will be jeopardized since pets (particularly dogs and cats) like to lick their owner, and the germs in the pet's mouth will be spread to human skins (including mouth, face, arms, etc). In some cases, pet owners have adverse reactions after being licked by their pets. Up to now, there is no feasible solution for the aforementioned problem.

Pet chew is a pet toy in the shape of a bone, a ball, a ring, a wood stick or a granule. A common pet chew is dog chew. Since dogs have the hobby of biting, the dog chew is generally designed in the shape suitable for the dogs to play. More importantly, there is also a dog chew specially designed for cleaning the chewed food of a pet's mouth. Chewing a dog chew frequently and brushing the dog's teeth periodically can retard the formation of dental plaque and tartar in a dog's mouth. As it is not easy to brush the teeth of your dog, the effect of brushing the dog's teeth regular is poor. Feeding a dog chew has a better effect than brushing the teeth and is capable of reducing gingivitis or the formation of other dirt or stain. The eating process is also a process of stabilizing the teeth and cleaning the mouth. Dogs have itching gums in their growth stage, feeding dogs with the dog chew has the effects of reducing pain, protecting home items from being damaged by the dogs, as well as cleaning the dog's mouth.

Conventional pet chews are mainly made of animal skins such as cow skins or pig skins and also mixed with starch, animal protein powder, and food fibers to form a branch shaped or spherical chews, or press-molded with rubber to form molded chews. However, these chews focus on the purpose of cleaning pet teeth only by using the rigidity of the chews to rub the pet teeth. Although this way can remove food residues and reduce the formation of dental calculus or plaque, it cannot overcome the problems regarding to the balance of oral flora and the breeding of germs, and cannot achieve a good effect for the pet's oral health. In addition, there is a conventional pet food added with probiotic such as bacillus or lactobacillus, but such pet food is intended to adjust the pet's intestinal health, and not designed for preventing oral diseases or the breeding or spreading of germs. When pets eat the pet food of this sort, the time for the pet food to be remained in the pet's mouth is less than 1 minute, and the probiotic does not have sufficient time to be activated or further reproduced and colonized, and thus failing to achieve a sufficient quantity or produce sufficient advantageous probiotic flora in the pet's mouth.

2. Summary of the Invention

Therefore, it is a primary objective of the present disclosure to provide a pet chew with a dental health function.

To achieve the aforementioned and other objectives, the present disclosure provides a pet chew with a dental health function, and the pet chew comprises a carrier; and a plurality of dental health members disposed on a surface or the interior of the carrier, and the dental health members contain a probiotic, a prebiotic, and an adhesive, and the carrier is made of starch, animal/plant protein powder, and bone powder.

Preferably, the probiotic includes bifidobacterium and/or lactobacillus and/or bacillus.

Preferably, the bifidobacterium includes but not limited to at least one selected from the group consisting of bifidobacterium bifidum, bifidobacterium animalis, bifidobacterium breve, bifidobacterium infantis, bifidobacterium adolescentis, and bifidobacterium longum.

Preferably, the lactobacillus includes but not limited to at least one selected from the group consisting of lactobacillus acidophilus, lactobacillus casei, lactobacilluscrispatus, lactobacillus bulgaricus, lactobacillus delbrueckii subsp. lactis, lactobacillus fermentum, lactobacillus gasseri, lactobacillus helveticus, lactobacillus johnsonii, lactobacillus paracasei, lactobacillus plantarum, lactobacillus reuteri, lactobacillus rhamnosus, and lactobacillus salivarius.

Preferably, the bacillus includes but not limited to at least one selected from the group consisting of bacillus coagulans, bacillus subtilis, and bacillus licheniformis.

Preferably, the probiotic is at least one selected from the group consisting of bifidobacterium, lactobacillus, and bacillus mixed according to any proportion.

Preferably, the medium and culture method of the probiotic are conventional medium and culture method in the related field.

Preferably, the probiotic is further added with an appropriate amount of freeze-dried protective agent, and the freeze-dried protective agent is provided for freeze-drying the activated bacteria sludge to obtain a probiotic (powder).

Preferably, the prebiotic includes but not limited to a functional oligosaccharide, and the functional oligosaccharide includes but not limited to at least one selected from the group consisting of isomaltooligosaccharide, fructooligosaccharide, galactooligosaccharide, xylooligosaccharide, lactosucrose, soybean oligosaccharide, and inulin.

Preferably, the adhesive is a plant-based adhesive and/or an animal-based adhesive, wherein the plant-based adhesive includes but not limited to starch, cellulose, tannin, gum arabic and sodium alginate, and the animal-based adhesive includes but not limited to bone glue, gelatin, blood albumin glue, casein, and shellac.

Preferably, the carrier is made of a material including but not limited to at least one selected from the group consisting of woven fabric, rubber, plastic, starch, protein, fiber, and bone powder, and the protein includes but not limited to plant protein, and animal skin.

Preferably, the pet chew has both flexibility and ductility, and the probiotic, prebiotic, and adhesive contained in the chew are released by the ductility of the chew during a pet's chewing process.

Preferably, the pet chew is calculated by the following mass percentages and composed of 0.1%-3% of probiotic powder, 1%-20% of prebiotic, 15%-40% of starch, 25%-45% of animal/plant protein powder, 5%-15% of bone powder, and 0.5%-10% of adhesive.

Preferably, the pet chew the pet chew is calculated by the following mass percentages and composed of 0.1%-1% of probiotic, 10%-20% of prebiotic, 20%-35% of starch, 30%-40% of animal/plant protein powder, 5%-15% of bone powder, and 0.5%-10% of adhesive.

The pet chew of the present invention is a pet toy with the dental health function and has both flexibility and ductility, so that the pet chew will not be cracked or broken easily during the chewing process, and the pet chew not just satisfies the pets' being fond of molar, and a pet usually chews the pet chew of the present invention for more than 3 minutes. In the meantime, the probiotic, prebiotic, and adhesive contained in the pet chew may be released by the ductility of the pet chew, so that the pet teeth can be cleaned for a long time during the chewing process. Further, the adhesive is mixed with the probiotic and the prebiotic, so that a large quantity of probiotic and prebiotic are attached to the pet's mouth. Since the prebiotic promotes the colonization of the probiotic, the advantageous probiotic group can be formed to inhibit the growth of germs inside the pet's mouth, so as to achieve good effects of maintaining the pet's oral health and eliminating the bad smell in the pet's mouth.

Preferably, the prebiotic is one selected from the group consisting of isomaltooligosaccharide, fructooligosaccharide, galactooligosaccharide, xylooligosaccharide, lactosucrose, soybean oligosaccharide, and inulin. The prebiotic promotes the beneficial bacteria to reproduce into an advantageous group, so as to achieve the effect of optimizing the balance of flora. The prebiotic may further be combined with bacteria cells of the same pathogenic source to weaken the adhesion of pathogenic bacteria to mucous membranes, so as to play a role in the prevention of infection.

The probiotic of the present invention comes from a human intestinal probiotic group and is a human beneficial probiotic. The probiotic is colonized in the pet's mouth during the chewing process of the pet chew and used to inhibit the germs harmful to human body. The probiotic not just maintains the pet's oral health only, but also reduces the risk of hurting the body of a pet owner after being licked by a pet (particularly a dog). Preferably, the probiotic is a bifidobacterium, a lactobacillus, and a bacillus such as lactobacillus reuteri, lactobacillus rhamnosus, lactobacillus salivarius, and bacillus coagulans. The probiotic is implanted into a pet's mouth and provided for inhibiting harmful microorganisms in the pet's mouth and maintaining the pet's oral health. Wherein, the lactobacillus salivarius can survive in the low pH environment produced by streptococcus mutan, so that the streptococcus mutan should be targeted and inhibited. The lactobacillus reuteri can secret reuterin and reutericyclin, and both of them have the broad-spectrum antibacterial effect which plays an important role of preventing dental caries and maintaining oral health. The bacillus coagulan is discovered to have important effects of effectively maintaining oral health, inhibiting the growth of streptococcus mutans, helping the immune system to enhance its function and resist pathogens, and reducing inflammatory cytokine.

The pet chew of the present invention comes with various different forms and shapes, and it can be a pet toy, or an item for protecting the pet's health.

In the pet chew of the present invention, a small amount of protein powder and bone powder may be added to improve the flavor. Without the need of supplying nutrition and energy to the pets, the effect of the probiotic can be provided. The pet chew not just provides chewing and grinding which are good to the pet's teeth only, but also achieves the effect of maintaining the pet's oral health.

### BRIEF DESCRIPTION OF THE DRAWINGS

FIG. 1A is a first perspective view of a pet chew product in accordance with a first preferred embodiment of the present invention;

FIG. 1B is a first cross-sectional view of a pet chew product in accordance with the first preferred embodiment of the present invention;

FIG. 2A is a second perspective view of a pet chew product in accordance with the second preferred embodiment of the present invention;

FIG. 2B is a second cross-sectional view of a pet chew product in accordance with the second preferred embodiment of the present invention;

FIG. 3A is a first perspective view of a pet chew product in accordance with a third preferred embodiment of the present invention;

FIG. 3B is a first cross-sectional view of a pet chew product in accordance with the third preferred embodiment of the present invention;

FIG. 4A is a second perspective view of a pet chew product in accordance with the fourth preferred embodiment of the present invention;

FIG. 4B is a second cross-sectional view of a pet chew product in accordance with the fourth preferred embodiment of the present invention,

FIG. 5 shows the growth result of probiotic combination co-cultivating with streptococcus mutan;

FIG. 6 shows the growth result of probiotic combination co-cultivating with porphyromonas gingivalis;

FIG. 7 shows the growth result of probiotic combination co-cultivating with fusobacterium nucleatum;

FIG. 8 shows the growth result of probiotic combination co-cultivating with tannerella forsythia; and

FIG. 9 shows the growth conditions of germs in the dog's mouth before and after the experiment.

### DESCRIPTION OF THE PREFERRED EMBODIMENTS

The above and other objects, features and advantages of this disclosure will become apparent from the following detailed description taken with the accompanying drawings. Only some embodiments of the present disclosure have been illustrated in the drawings, but it should be pointed out that many other modifications are conceivable within the scope of the following claims.

### Preferred Embodiment 1

A pet chew with a dental health function 1 of this embodiment comprises a carrier 11 and a plurality of dental health members 12, and the dental health member 12 contains a probiotic, a prebiotic, and an adhesive. The dental health member is composed of 0.1% of probiotic, 1% of isomaltooligosaccharide, 10% of cellulose, 20% of gelatin, 23.9% of blood albumin glue, 40% of starch, and 5% of bone powder, wherein all of the above ingredients are calculated by mass percentage.

Wherein, the probiotic is composed of bifidobacterium bifidum, bifidobacterium breve, lactobacillus acidophilus, lactobacilluscrispatus, lactobacillus delbrueckii subsp. lactis, bacillus coagulans, bacillus subtilis, and bacillus licheniformis.

The bifidobacterium probiotic (including bifidobacterium bifidum, and bifidobacterium breve) is prepared by an inoculation volume of 5% of bifidobacterium bifidum and bifidobacterium breve added into a BS liquid medium respectively and cultured at 37° C. for 48 hours, and the bacteria are collected, and the live bacteria counts of the bifidobacterium bifidum and bifidobacterium breve are 1.0-5.0×10⁸ cfu/g respectively.

The lactobacillus probiotic (including lactobacillus acidophilus, lactobacilluscrispatus, and lactobacillus delbrueckii subsp. lactis) is prepared by an inoculation volume of 5% of lactobacillus acidophilus, lactobacillus casei, lactobacillus fermentum, and lactobacillus salivarius added into a MRS liquid medium respectively and cultured at 37° C. for 48 hours, and the bacteria are collected, and the live bacteria counts of the lactobacillus acidophilus, lactobacilluscrispatus, and lactobacillus delbrueckii subsp. lactis are 1.0-5.0×10⁸ cfu/g respectively.

The bacillus probiotic (including bacillus coagulans, bacillus subtilis, bacillus licheniformis) is prepared by adding an inoculation volume of 5% of bacillus coagulans, bacillus subtilis, and bacillus licheniformis added into a LB medium and cultured at 37° C. for 48 hours, and the bacteria are connected, and the live bacteria counts of the bacillus coagulans, bacillus subtilis, and bacillus licheniformis are 1.0-5.0×10⁸ cfu/g respectively.

The bifidobacterium bifidum, bifidobacterium breve, lactobacillus acidophilus, lactobacilluscrispatus, lactobacillus delbrueckii subsp. lactis, bacillus coagulans, bacillus subtilis, and bacillus licheniformis are mixed according to a mass proportion including but not limited to (1:1:1:1:1:1:1:1) to obtain a probiotic sludge, and the probiotic sludge is mixed with a freeze-dried protective agent according to the mass ratio of 1:1 and then freeze-dried to obtain a probiotic powder.

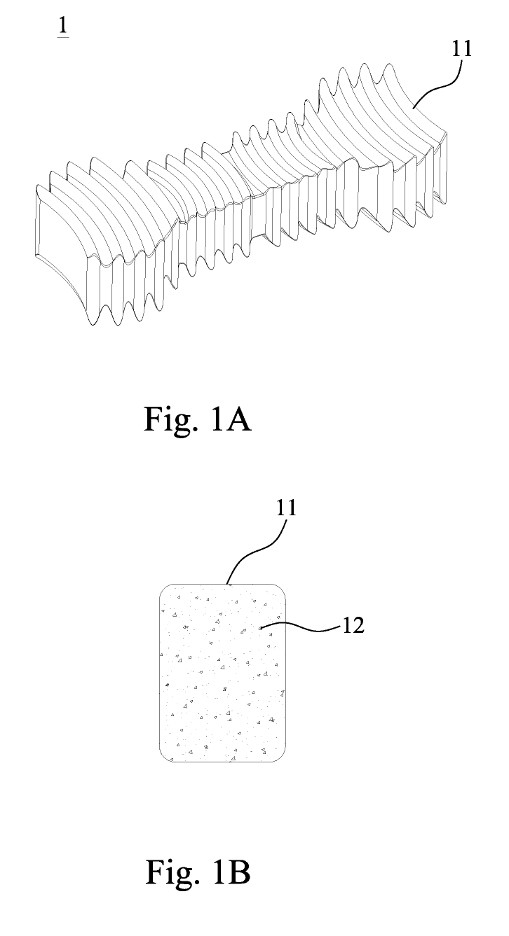

In the preparation method, the ingredients of the composition of the pet chew are weighed, and then mixed uniformly, and finally molded into the pet chew with a dental health function. With reference to FIGS. 1A and 1B for the pet chew product of the present invention, the dental health members 12 are scattered in the carrier 11 uniformly.

### Preferred Embodiment 2

A pet chew with a dental health function 1 of this embodiment is composed of the following ingredients calculated by mass percentage: 0.3% of probiotic, 8% of fructooligosaccharide, 10% of galactooligosaccharide, 0.5% of bone glue, 20% of tannin, 18.2% of gum arabic, 37% of starch, and 6% of bone powder.

The activated probiotic powder includes bifidobacterium adolescentis, bifidobacterium longum, lactobacillus gasseri, lactobacillus helveticus, lactobacillus johnsonii, bacillus coagulans, and bacillus subtilis mixed according to the mass percentages and their live bacteria counts are 1.0-5.0×10⁸ cfu/g respectively, and the probiotic is obtained by mixing the mixture with a freeze-dried protective agent according to the mass ratio of 1:1 and then freeze-drying the mixture.

Wherein, the culture method of the probiotic is the same as the culture method of Preferred Embodiment 1.

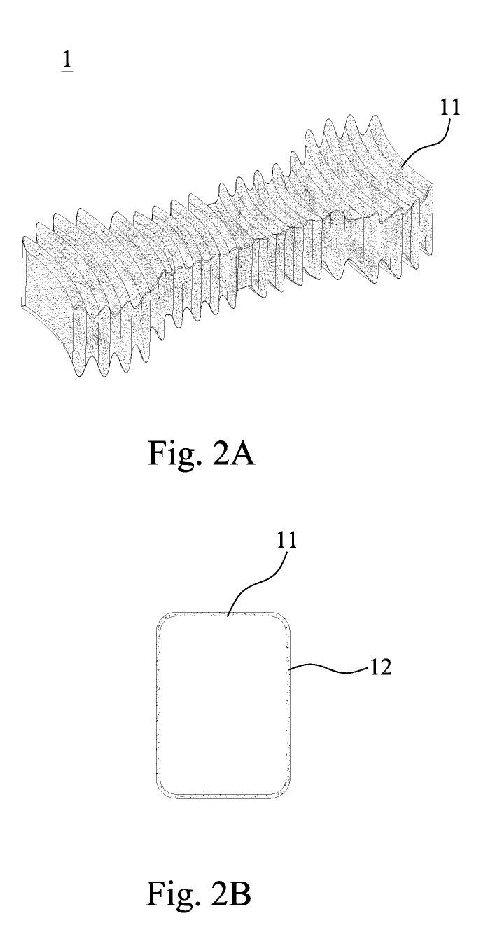

In the preparation method, the ingredients of the pet chew other than the probiotic, prebiotic, and adhesive are mixed uniformly, and then molded, and finally a mixture of probiotic, prebiotic and adhesive is coated onto the surface of the molded pet chew to obtain the pet chew 1 with the dental health function. With reference to FIGS. 2A and 2B for the pet chew product of the present invention, the dental health members 12 are attached onto the surface of the carrier 11 by the effect of the adhesive.

### Preferred Embodiment 3

A pet chew with a dental health function 1 is composed of the following ingredients calculated by mass percentage: 0.5% of probiotic, 3% of xylooligosaccharide, 9.5% of sodium alginate, 20% of casein, 25% of shellac, 35% of starch, and 7% of bone powder.

The activated probiotic powder is composed of bifidobacterium animalis, bifidobacterium infantis, lactobacillus paracasei, lactobacillus bulgaricus, lactobacillus casei, lactobacillus fermentum, bacillus coagulans, and bacillus licheniformis mixed according their mass percentages, and their live bacteria counts are 1.0-5.0×10⁸ cfu/g respectively, and the probiotic is obtained by mixing the mixture with a freeze-dried protective agent according to the mass ratio of 1:1, and then freeze-drying the mixture.

Wherein, the culture method of the probiotic is the same as the culture method of Preferred Embodiment 1.

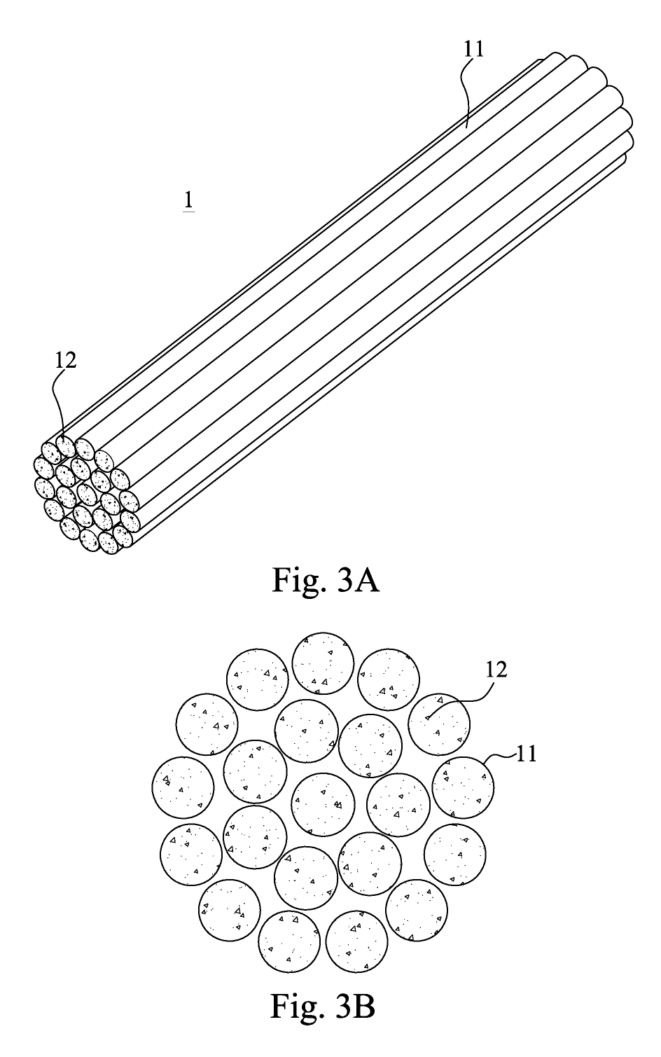

In the preparation method, the probiotic, prebiotic, adhesive, and other ingredients are mixed uniformly, and then molded into a pet chew with a dental health function 1. With reference to FIGS. 3A and 3B for the pet chew product of the present invention, the dental health members 12 are scattered into the carrier 11 uniformly.

### Preferred Embodiment 4

A pet chew with a dental health function 1 is composed of the following ingredients calculated by mass percentage: 0.7% of probiotic, 8% of lactosucrose, 8% of soybean oligosaccharide, 2% of gelatin, 20% of cellulose, 23.3% of sodium alginate, 30% of starch, and 8% of bone powder.

The activated probiotic powder includes bifidobacterium bifidum, bifidobacterium adolescentis, lactobacillus acidophilus, lactobacillus gasseri, lactobacillus paracasei, bacillus subtilis, and bacillus licheniformis mixed according to their mass percentages and their live bacteria counts are 1.0-5.0×10⁸ cfu/g respectively, and the probiotic power is obtained by mixing the mixture with a freeze-dried protective agent according to the mass ratio of 1:1 and then freeze-drying the mixture.

Wherein, the culture method of the probiotic is the same as the culture method of Preferred Embodiment 1.

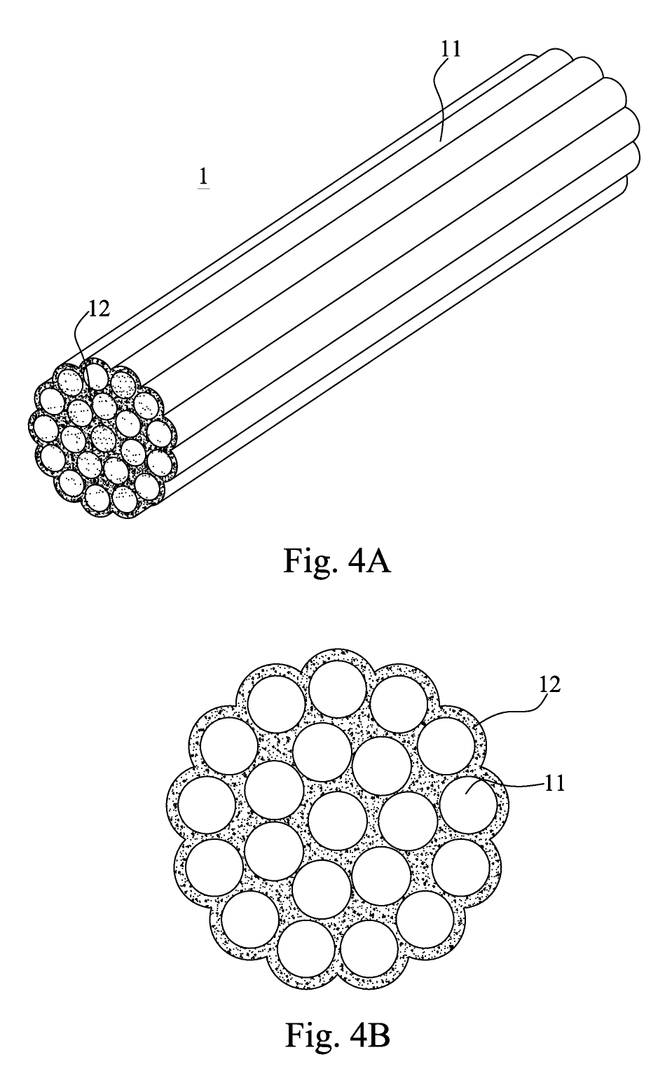

In the preparation method, the ingredients other than the probiotic, prebiotic, and adhesive are mixed uniformly and then molded, and finally a mixture of the probiotic, prebiotic, and adhesive is coated onto a surface of the pet chew to obtain the pet chew 1 with a dental health function. With reference to FIGS. 4A and 4B, the dental health members 12 are attached onto the surface of the carrier 11 by the effect of the adhesive.

### Preferred Embodiment 5

A pet chew with a dental health function 1 is composed of the following ingredients calculated by mass percentage: 0.9% of probiotic, 5% of inulin, 2.5% of gum arabic, 20% of bone glue, 20.6% of gelatin, 22% of starch, 9% of bone powder, and 20% of rubber.

The activated probiotic powder includes bifidobacterium bifidum, bifidobacterium longum, lactobacilluscrispatus, lactobacillus helveticus, lactobacillus bulgaricus, bacillus coagulans, bacillus subtilis, and bacillus licheniformis mixed according to their mass percentages, and their live bacteria counts are 1.0-5.0×10⁸ cfu/g respectively, and the mixture is mixed with a freeze-dried protective agent according to the mass ratio of 1:1 and then freeze-drying the mixture.

Wherein, the culture method of the probiotic is the same as the culture method of Preferred Embodiment 1.

In the preparation method, the probiotic, prebiotic, and adhesive other than rubber are mixed in advance, and then coated onto the surface of the carrier made of rubber to obtain the pet chew with a dental health function.

### Preferred Embodiment 6

A pet chew with a dental health function 1 is composed of the following ingredients calculated by mass percentage: 2.4% of probiotic, 7% of isomaltooligosaccharide, 7% of fructooligosaccharide, 7% of blood albumin glue, 15% of cellulose, 15.6% of tannin, 32% of starch, and 14% of bone powder.

The activated probiotic powder includes bifidobacterium bifidum, bifidobacterium animalis, German lactobacillus, lactobacillus johnsonii, lactobacillus casei, bacillus coagulans, and bacillus subtilis mixed according to their mass percentage, and their live bacteria counts are 1.0-5.0×10⁸ cfu/g respectively, and the probiotic is obtained by mixing the mixture with a freeze-dried protective agent according to the mass ratio of 1:1, and finally freeze-drying the mixture.

Wherein, the culture method of the probiotic is the same as the culture method of Preferred Embodiment 1.

In the preparation method, the ingredients of the ingredients of the pet chew are weighed and mixed uniformly, and then molded into a pet chew with a dental health function 1.

### Preferred Embodiment 7

A pet chew with a dental health function 1 is composed of the following ingredients calculated by mass percentage: 1.7% of probiotic, 8% of galactooligosaccharide, 4% of tannin, 20% of blood albumin glue, 20.3% of casein, 35% of starch, and 11% of bone powder.

The activated probiotic powder includes bifidobacterium bifidum, bifidobacterium infantis, lactobacillus gasseri, lactobacillus fermentum, lactobacillus salivarius, bacillus coagulans, and bacillus licheniformis mixed uniformly according to their mass percentages, and their live bacteria counts are 1.0-5.0×10⁸ cfu/g respectively, and the probiotic is obtained by mixing the mixture with a freeze-dried protective agent according to the mass ratio of 1:1 and freeze-drying the mixture.

Wherein, the culture method of the probiotic is the same as the culture method of Preferred Embodiment 1.

In the preparation method, the ingredients other than the probiotic, prebiotic, and adhesive are mixed uniformly, and then molded, and finally a mixture of the probiotic, prebiotic, and adhesive is coated onto a surface of the pet chew to obtain the pet chew with an oral health.

### Preferred Embodiment 8

A pet chew with a dental health function 1 is composed of the following ingredients calculated by mass percentage: 2% of probiotic, 5% of xylooligosaccharide, 5% of lactosucrose, 5% of casein, 20% of gum arabic, 23% of sodium alginate, 28% of starch, and 12% of bone powder.

The activated probiotic powder includes bifidobacterium bifidum, bifidobacterium breve, bifidobacterium adolescentis, lactobacillus gasseri, lactobacillus bulgaricus, lactobacillus reuteri, bacillus subtilis, and bacillus licheniformis mixed according to their mass percentages, and their live bacteria counts are 1.0-5.0×10⁸ cfu/g respectively, and the probiotic is obtained by mixing the mixture with a freeze-dried protective agent according to the mass ratio of 1:1, and freeze-drying the mixture.

Wherein, the culture method of the probiotic is the same as the culture method of Preferred Embodiment 1.

In the preparation method, the ingredients are weighed and mixed uniformly, and then molded into the pet chew with a dental health function.

### Preferred Embodiment 9

A pet chew with a dental health function 1 is composed of the following ingredients calculated by mass percentage: 2.2% of probiotic, 6% of soybean oligosaccharide, 6% of inulin, 6% of shellac, 17% of cellulose, 20% of gum arabic, 30% of starch, 13% of bone powder.

The activated probiotic powder includes bifidobacterium breve, bifidobacterium longum, lactobacillus helveticus, lactobacillus casei, lactobacillus rhamnosus, bacillus coagulans, and bacillus subtilis mixed according to their mass percentages, and their live bacteria counts are 1.0-5.0×10⁸ cfu/g respectively, and the probiotic is obtained by mixing the mixture with a freeze-dried protective agent according to the mass ratio of 1:1, and then freeze-drying the mixture.

Wherein, the culture method of the probiotic is the same as the culture method of Preferred Embodiment 1.

In the preparation method, the ingredients other than the probiotic, prebiotic, and adhesive are mixed uniformly, and then molded, and finally a mixture of the probiotic, prebiotic, and adhesive is coated onto a surface of the pet chew to obtain the pet chew with a dental health function.

### Preferred Embodiment 10

A pet chew with a dental health function 1 is composed of the following ingredients calculated by mass percentage: 1.4% of probiotic, 7% of fructooligosaccharide, 3% of cellulose, 15% of bone glue, 20% of shellac, 24% of starch, 10% of bone powder, and 20% of woven fabric.

The activated probiotic powder includes bifidobacterium breve, bifidobacterium animalis, lactobacillus johnsonii, lactobacillus fermentum, lactobacillus plantarum, bacillus coagulans, and bacillus licheniformis mixed according to their mass percentages, and their live bacteria counts are 1.0-5.0×10⁸ cfu/g respectively, and the probiotic is obtained by mixing the mixture with a freeze-dried protective agent according to the mass ratio of 1:1, and then freeze-drying the mixture.

Wherein, the culture method of the probiotic is the same as the culture method of Preferred Embodiment 1.

In the preparation method, the probiotic, prebiotic, and adhesive other than woven fabric are mixed uniformly, and then coated onto a surface of the carrier made of the woven fabric to obtain the pet chew with a dental health function.

### Preferred Embodiment 11

A pet chew with a dental health function 1 is composed of the following ingredients calculated by mass percentage: 2.6% of probiotic, 7% of galactooligosaccharide, 8% of xylooligosaccharide, 8% of tannin, 5% of bone glue, 10% of blood albumin glue, 10% of casein, 34% of starch, and 15% of bone powder.

The activated probiotic powder includes bifidobacterium breve, bifidobacterium infantis, lactobacillus acidophilus, lactobacillus helveticus, lactobacillus fermentum, lactobacillus salivarius, and bacillus coagulans mixed according to their mass percentages and their live bacteria counts are 1.0-5.0×10⁸ cfu/g respectively, and the probiotic is obtained by mixing the mixture with a freeze-dried protective agent according to the mass ratio of 1:1, and then freeze-drying the mixture.

Wherein, the culture method of the probiotic is the same as the culture method of Preferred Embodiment 1.

In the preparation method, the ingredients of the pet chew are weighed according to the mass percentage, and then mixed uniformly, and finally molded into the pet chew with a dental health function.

### Preferred Embodiment 12

A pet chew with a dental health function 1 is composed of the following ingredients calculated by mass percentage: 3% of probiotic, 5% of lactosucrose, 5% of soybean oligosaccharide, 10% of inulin, 10% of gelatin, 10% of cellulose, 10% of gum arabic, 17% of sodium alginate, 15% of starch, and 15% of bone powder.

The activated probiotic powder includes bifidobacterium adolescentis, bifidobacterium animalis, lactobacilluscrispatus, lactobacillus johnsonii, lactobacillus reuteri, and bacillus subtilis mixed according to their mass percentages, and the live bacteria counts are 1.0-5.0×10⁸ cfu/g respectively, and the probiotic is obtained by mixing the mixture with a freeze-dried protective agent according to the mass ratio of 1:1, and then freeze-drying the mixture.

Wherein, the culture method of the probiotic is the same as the culture method of Preferred Embodiment 1.

In the preparation method, the ingredients other than the probiotic, prebiotic, and adhesive are mixed uniformly, and then molded, and finally a mixture of the probiotic, prebiotic, and adhesive is coated onto a surface of the pet chew to obtain the pet chew with a dental health function.

### Preferred Embodiment 13

A pet chew with a dental health function 1 is composed of the following ingredients calculated by mass percentage: 2.8% of probiotic, 6% of lactosucrose, 7% of blood albumin glue, 10% of tannin, 18.2% of sodium alginate, 27% of starch, 9% of bone powder, and 20% of a pet toy sold on the market.

The activated probiotic powder includes bifidobacterium longum, bifidobacterium animalis, bifidobacterium infantis, lactobacillus delbrueckii subsp. lactis, lactobacillus bulgaricus, lactobacillus rhamnosus, and bacillus licheniformis mixed according to their mass percentages, and their live bacteria counts are 1.0-5.0×10⁸ cfu/g respectively, and the probiotic powder is obtained by mixing the mixture with a freeze-dried protective agent according to the mass ratio of 1:1 and then freeze-drying the mixture.

Wherein, the culture method of the probiotic is the same as the culture method of Preferred Embodiment 1.

In the preparation method, the probiotic, prebiotic, and adhesive other than the pet toy sold on the market are mixed in advance, and then coated onto a surface of the commercially available pet toy to obtain the pet chew with a dental health function.

To test the effect of the pet chew of the present invention on the recovery of oral diseases, a strain co-cultivation experiment and an experiment on a dog's recovery from the periodontal disease are conducted to verify the efficacy of the pet chew of the present invention for periodontal diseases.

1. Study on Antibacterial Properties

Dilute the cultures of the germs streptococcus mutan, porphyromonas gingivalis, fusobacterium nucleatum, and tannerella forsythia causing the periodontal disease to 10⁶ CFU/mL. Coat 0.1 mL of the cultures onto a plate with a beef extract peptone medium. Place an Oxford cup (having a diameter of 8 mm) onto the plate.

Add 100 μL, of probiotic combination bacilli according to Preferred Embodiment 1, and culture the mixture at 37° C. for 24 hours. Measure the size of the antibacterial ring. Use sterile saline as the control. Experiment results are listed in Table 1 below.

TABLE 1
Diameter of the Antibacterial Ring (Unit: mm)

| | Streptococcus | Porphyromonas | Fusobacterium | Tannerella |
|---|---|---|---|---|
| | mutan | gingivalis | nucleatum | forsythia |
| Exper- | 14.3 ± 1.1 | 19.5 ± 0.6 | 17.3 ± 1.7 | 16.1 ± 1.0 |
| iment | | | | |
| Group | | | | |
| Control | — | — | — | — |
| Group | | | | |

Note:
stands for no anti-bacterial effect

The experiment results show that the probiotic combination has a very good effect of inhibiting four pathogens, respectively: streptococcus mutan, porphyromonas gingivalis, fusobacterium nucleatum, and tannerella forsythia.

2. Co-Cultivation Experiment of Strains

The probiotic combination of Preferred Embodiment 1 is added into the liquid medium of streptococcus mutan, porphyromonas gingivalis, fusobacterium nucleatum, and tannerella forsythia separately, and the media are co-cultivated at 37° C., and samples are taken at different cultivation time. A plate count method is used to learn the antibacterial condition of the probiotic to the pathogen.

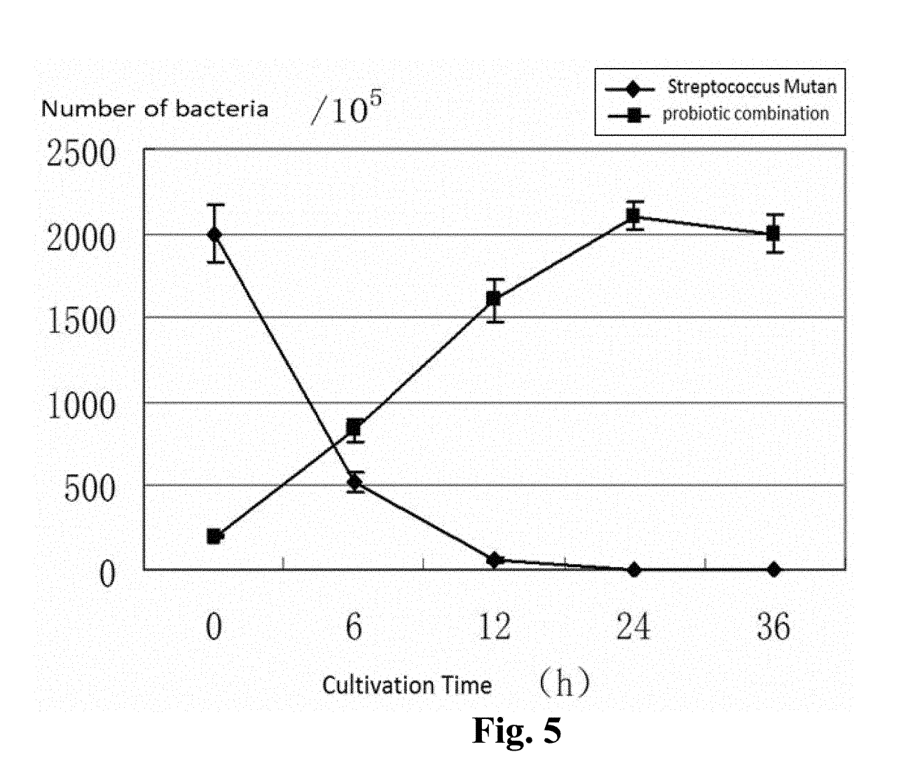

The probiotic combination is co-cultivated with the streptococcus mutan, and the growth condition is shown in FIG. 5.

After the probiotic combination and the streptococcus mutan have been co-cultivated for 36 hours, no streptococcus mutan exist. It shows that the probiotic combination has the effect of inhibiting the streptococcus mutan.

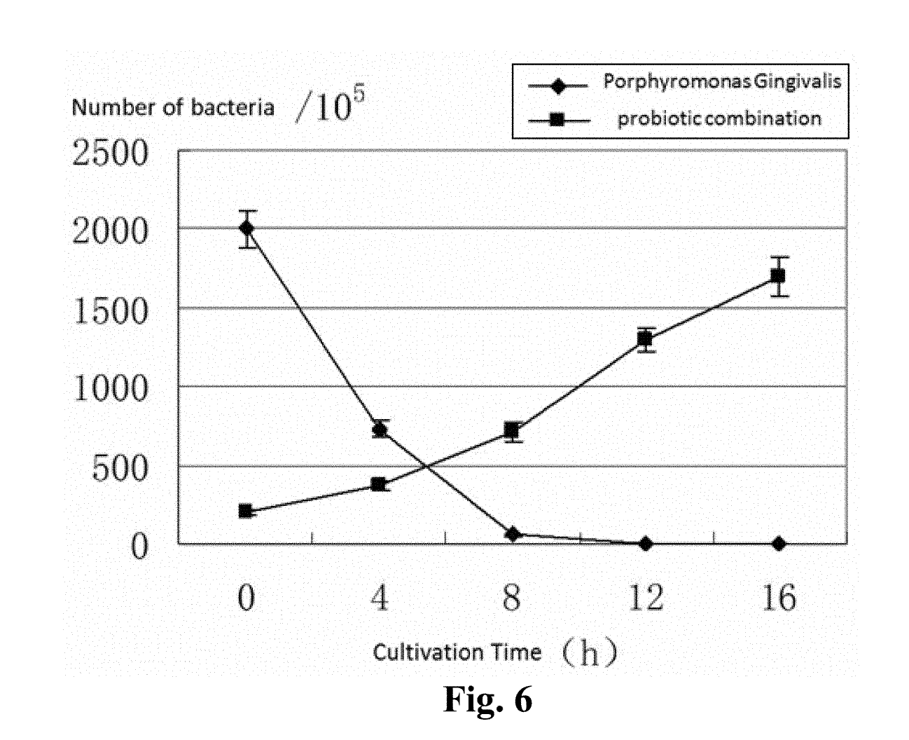

The probiotic combination and the porphyromonas gingivalis are co-cultivated, the growth condition is shown in FIG. 6.

After the probiotic combination and the porphyromonas gingivalis have been co-cultivated for 16 hours, no porphyromonas gingivalis exists. It shows that the probiotic combination has the effect of inhibiting the porphyromonas gingivalis.

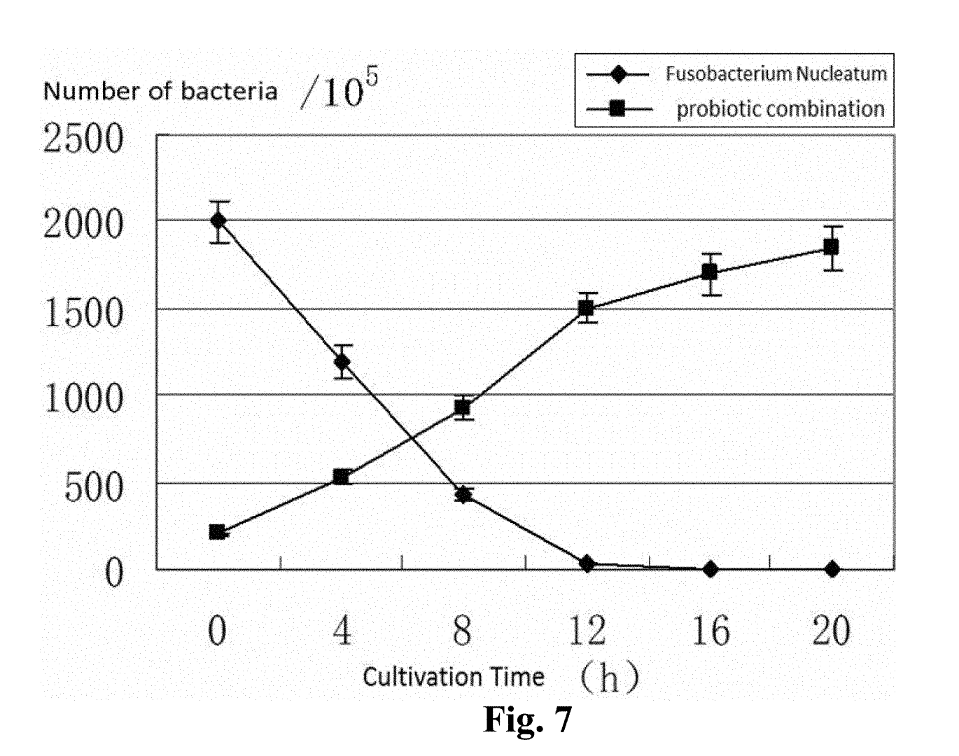

The probiotic combination and the fusobacterium nucleatum are co-cultivated, and the growth condition is shown in FIG. 7.

After the probiotic combination and the fusobacterium nucleatum have been co-cultivated for 16 hours, no fusobacterium nucleatum exists. It shows that the probiotic combination has the effect of inhibiting the fusobacterium nucleatum.

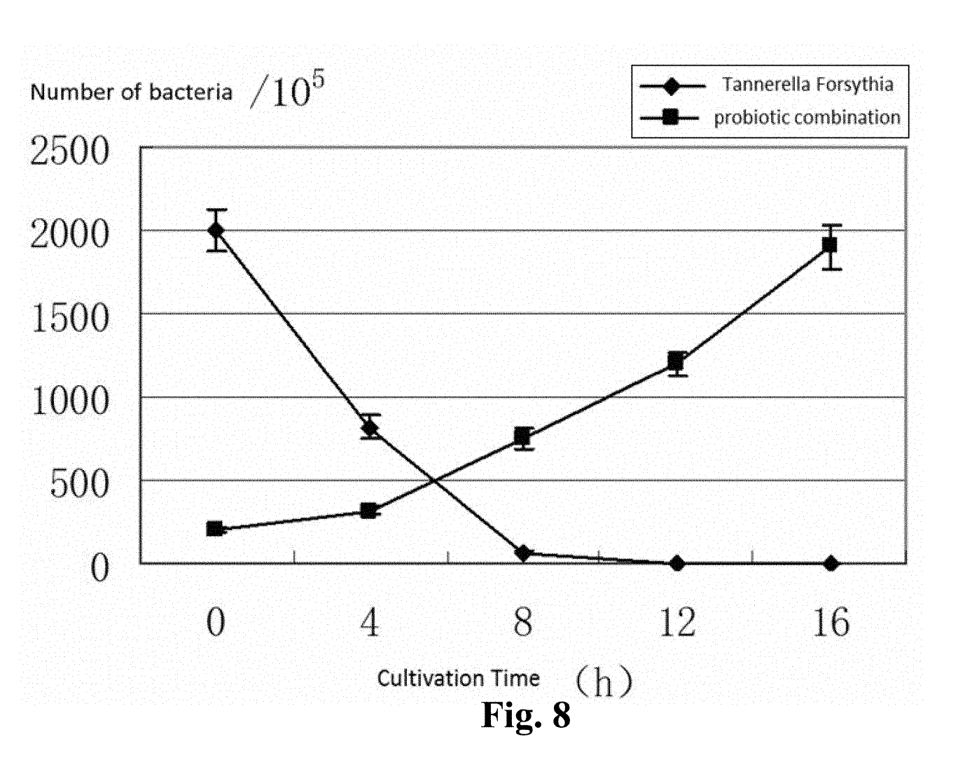

The probiotic combination and the tannerella forsythia are co-cultivated, and the growth condition is shown in FIG. 8.

After the probiotic combination and the tannerella forsythia have been co-cultivated for 20 hours, no tannerella forsythia exists. It shows that the probiotic combination has the effect of inhibiting the tannerella forsythia.

3. Experiment on the Recovery of Dog's Periodontal Disease

Five groups of three-year old dogs suffering periodontal diseases and having bad breath are taken for experiments, wherein each group includes three of these dogs.

Experiment Group 1: The pet chew according to Preferred Embodiment 1 is given to the dogs of this group daily;

Experiment Group 2: The pet chew according to Preferred Embodiment 2 is given to the dogs of this group daily;

Experiment Group 3: The pet chew according to Preferred Embodiment 5 is given to the dogs of this group daily;

Experiment Group 4: The pet chew according to Preferred Embodiment 13 is given to the dogs of this group daily;

Experiment Group 5: The pet chew according to Preferred Embodiment 5 excluding the ingredients of probiotic, prebiotic and adhesive is given to the dogs of this group daily, wherein the formula just contains starch and cow bone powder only;

Experiment Group 6: a dog chew commercially available on the market and using pig skin as the raw material is given to the dogs of this group daily;

Experiment Group 7: a dog chew commercially available on the market and using rubber as the raw material is given to the dogs of this group daily;

Experiment Group 8: a dog food commercially available on the market and containing lactobacillus is given to the dogs of this group daily;

The dogs of the above experiment groups are fed for 14 consecutive days, and sterile filter paper with the size of 1 cm×1 cm is used for collecting bacteria from the oral periodontal tissues of the dogs of five groups at the beginning and the end of the experiment respectively, and the bacteria are submerged into 1.0 mL of physiological saline, and then diluted according to the decimally serially dilution, and the plate count method is used for calculating the number of colonies, and the growth conditions of bacteria in the dogs' mouths of both Experiment Group and Control Group are observed, while determining whether or not the bad breath of the dogs has been eliminated before and after the experiment.

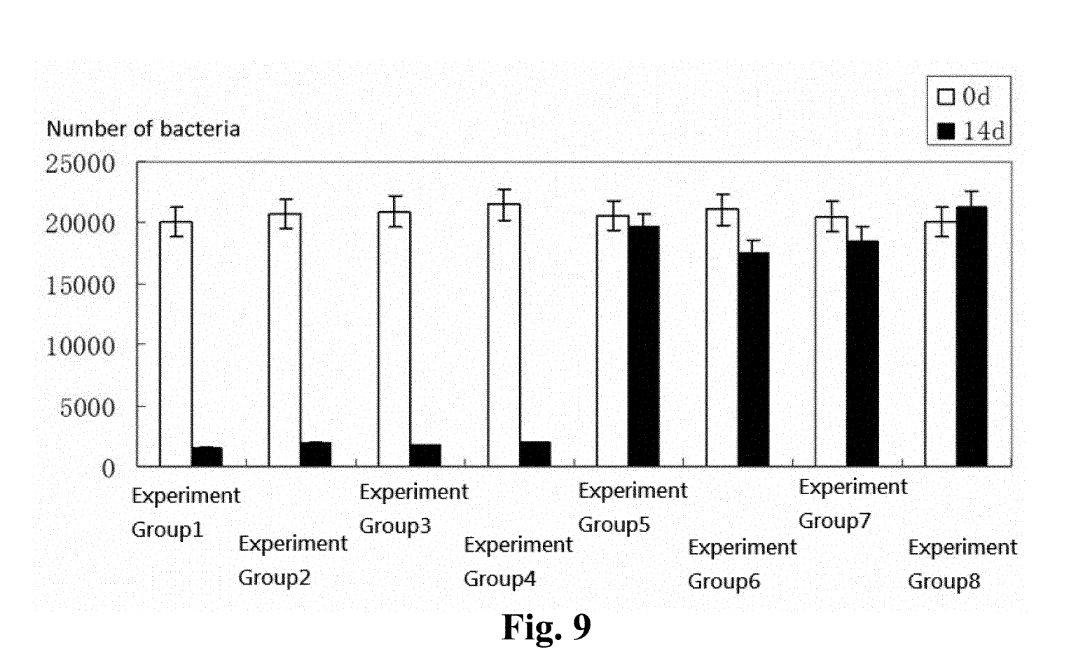

The growth conditions of germs in the dog's mouth before and after the experiment are shown in FIG. 9.

The result above shows that after feeding the dogs for 14 days, the periodontal disease of the dogs of Experiment Groups 1, 2, 3 and 4 is improved significantly, and the decrease of number of germs in the mouth of the dogs of Experiment Groups 5, 6, and 7 is insignificant, and the number of germs in the mouth of the dogs of Experiment Group 8 increases slightly. This experiment result shows that the pet chew of the present invention has a significant effect of improving the dogs' periodontal disease.

Comparison of Stinky Smell in Dog's Mouth Before and After the Experiment is given in Table 2 below.

TABLE 2
Determination Result of Dog's Oral Malodor Before and After Experiment

| Cultivation | Experiment | Experiment | Experiment | Experiment | Experiment | Experiment | Experiment | Experiment |
|---|---|---|---|---|---|---|---|---|
| time (d) | Group 1 | Group 2 | Group 3 | Group 4 | Group 5 | Group 6 | Group 7 | Group 8 |
| 0 | + | + | + | + | + | + | + | + |
| 14 | −− | −− | −− | −− | + | + | + | + |

Note:
+ stands for bad breath, and
− stands for no bad breath

The above experiment result shows that after 14 days of feeding, the bad breath of the dogs of the Experiment Groups 1, 2, 3 and 4 disappears, and the bad breath of the dogs of the Experiment Groups 5, 6, 7, and 8 still exists. The experiment result shows that the pet chew of the present invention has a significant effect of improving the dogs' bad breath.

4. Chewing Time Comparison Experiment

Five groups of three-year old dogs without suffering from periodontal disease are used for experiment, wherein each group includes three dogs.

Experiment Group 1: The pet chew according to Preferred Embodiment 1 is given to the dogs of this group daily;

Experiment Group 2: The pet chew according to Preferred Embodiment 2 is given to the dogs of this group daily;

Experiment Group 3: The pet chew according to Preferred Embodiment 5 is given to the dogs of this group daily;

Experiment Group 4: The pet chew according to Preferred Embodiment 13 is given to the dogs of this group daily;

Experiment Group 5: The pet chew according to Preferred Embodiment 5 excluding the ingredients of probiotic, prebiotic and adhesive is given to the dogs of this group daily, wherein the formula just contains starch and cow bone powder only;

Experiment Group 6: a dog chew commercially available on the market and using pig skin as the raw material is given to the dogs of this group daily;

Experiment Group 7: a dog chew commercially available on the market and using rubber as the raw material is given to the dogs of this group daily;

Experiment Group 8: a dog food commercially available on the market and containing lactobacillus is given to the dogs of this group daily;

The chew or dog food of the aforementioned Experiment Groups is set to equal size and volume, and the average of the dog's biting time is taken, and the results are listed in Table 3.

TABLE 3
Chewing Time Comparison Experiment

| Cultivation | Experiment | Experiment | Experiment | Experiment | Experiment | Experiment | Experiment | Experiment |
|---|---|---|---|---|---|---|---|---|
| time (d) | Group 1 | Group 2 | Group 3 | Group 4 | Group 5 | Group 6 | Group 7 | Group 8 |
| Chewing | 3.6 | 3.4 | 3.5 | 3.7 | 3.0 | 2.5 | 2.9 | <1 |
| Time (min) | | | | | | | | |

The experiment result listed in Table 3 shows that the chewing time of the pet chew of the present invention by the dogs in the Experiment Groups 1, 2, 3 and 4 exceeds 3 minutes, so that the prebiotic and probiotic are adhered onto the surface of the dogs' oral cavity or teeth through the effect of the adhesive. In addition, the prebiotic promotes the colonization of the probiotic and the formation of the advantageous probiotic group, so as to inhibit the growth of germs in the dogs' mouth and achieve the effects of maintaining good dental health, and eliminating bad breath. Since the pet chew given to the dogs of the Experiment Group 5 is lack of the composition of dental health members, therefore the flexibility and ductility of the pet chew are affected, and the dog's biting time is shortened relatively. Since the dog chew given to the dogs of the Experiment Groups 6 and 7 is primarily made of pig skin, and rubber, the dog chew has a specific hardness, and the dog's biting time is around 2.5-3 minutes. However, the pet chew given to the dogs of the Experiment Groups 6 and 7 has no dental health member with the dental health function, so that such pet chews do not have the dental health effect of the pet chew of the present invention. The dog food given to the dogs of the Experiment Group 8 is a pet food similar to most of the conventional pet food, so that the dog's biting time is relatively shorter (which is less than 1 minute), and the probiotic has no sufficient time to be activated or further reproduced and colonized. Such pet food fails to form sufficient advantageous probiotic flora in the dog's mouth.

What is claimed is:

1. A pet chew with a dental health function, comprising:
a carrier; and
a plurality of dental health members disposed on a surface or the interior of the carrier, and containing a probiotic, a prebiotic, and an adhesive.

2. The pet chew of claim 1, wherein the probiotic is one selected from the group consisting of a human intestinal probiotic group, and a human beneficial probiotic including a bifidobacterium and/or a lactobacillus and/or a bacillus.

3. The pet chew of claim 2, wherein the bifidobacterium is one selected from the group consisting bifidobacterium bifidum, bifidobacterium animalis, bifidobacterium breve, bifidobacterium infantis, bifidobacterium adolescentis, and bifidobacterium longum.

4. The pet chew of claim 2, wherein the lactobacillus is one selected from the group consisting of lactobacillus acidophilus, lactobacillus casei, lactobacilluscrispatus, lactobacillus bulgaricus, lactobacillus delbrueckii subsp. lactis, lactobacillus fermentum, lactobacillus gasseri, lactobacillus helveticus, lactobacillus johnsonii, lactobacillus paracasei, lactobacillus plantarum, lactobacillus reuteri, lactobacillus rhamnosus, and lactobacillus salivarius.

5. The pet chew of claim 2, wherein the bacillus is one selected from the group consisting of bacillus coagulans, bacillus subtilis, and bacillus licheniformis.

6. The pet chew of claim 1, wherein the prebiotic includes a functional oligosaccharide selected from the group consisting of isomaltooligosaccharide, fructooligosaccharide, galactooligosaccharide, xylooligosaccharide, lactosucrose, soybean oligosaccharide, and inulin.

7. The pet chew of claim 1, wherein the adhesive is a plant-based adhesive and/or an animal-based adhesive, and the plant-based adhesive includes starch, cellulose, tannin, gum arabic and sodium alginate, and the animal-based adhesive includes bone glue, gelatin, blood albumin glue, casein, and shellac.

8. The pet chew of claim 1, wherein the carrier is made of a material selected from the group of woven fabric, rubber, plastic, starch, protein, fiber, and bone powder or a mixture thereof.

9. The pet chew of claim 1, wherein the pet chew has both flexibility and ductility, and the probiotic, prebiotic, and adhesive contained in the chew are released by the ductility of the chew during a pet's chewing process.

10. The pet chew according to claim 1, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

11. The pet chew according to claim 2, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

12. The pet chew according to claim 3, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

13. The pet chew according to claim 4, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

14. The pet chew according to claim 5, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

15. The pet chew according to claim 6, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

16. The pet chew according to claim 7, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.

17. The pet chew according to claim 8, wherein the pet chew is composed of 0.1%-3% of probiotic, 1%-25% of prebiotic, 5%-40% of starch, 1%-10% of animal/plant protein powder, 1%-15% of bone powder, and 0.5%-15% of adhesive.
